(19) United States
(12) Patent Application Publication
(10) Pub. No.: US 2024/0003812 A1
(43) Pub. Date: Jan. 4, 2024

(54) MOBILE PHONE-BASED BIOLOGICAL TESTING METHOD AND APPARATUS

(71) Applicant: FACON CO., LTD., Suzhou (CN)

(72) Inventors: Feng WANG, Suzhou (CN); Wenqing YAN, Suzhou (CN); Xiang ZHAN, Suzhou (CN); Xingcan DAI, Suzhou (CN)

(73) Assignee: FACON CO., LTD., Suzhou, JS (CN)

(21) Appl. No.: 18/037,733

(22) PCT Filed: Dec. 3, 2021
(86) PCT No.: PCT/CN2021/135273

§ 371 Date: May 18, 2023

(30) Foreign Application Priority Data
Dec. 24, 2020 (CN) 202011554219.8

(51) Int. Cl. G01N 21/64

(52) U.S. Cl. CPC G01N 21/6456; G01N 21/6428; G01N 2021/6478; G01N 2021/6471; G01N 2021/6423

(57) ABSTRACT

Provided are a mobile phone-based biological testing method and apparatus. The method includes: selecting N kinds of quantum dots of specific wavelengths to label N to-be-tested biomarkers in a sample, where N is 1, 2 or 3; exciting to-be-tested biomarker-quantum dot compounds using excitation light; photographing the above excited compounds for multiple times, so as to obtain a first original image file in a RAW format with a 16-bit depth; photographing unlabeled quantum dots to obtain a second original image file in a RAW format with a 16-bit depth; calculating a matrix of transition coefficients of fluorescence intensities of the quantum dots received by channels in the second original image file; calculating a fluorescence intensity of each of the to-be-tested biomarker-quantum dot compounds in the first original image file; and obtaining a concentration of each of the to-be-tested biomarkers according to the fluorescence intensity, so as to complete biological testing.

### CROSS REFERENCE TO RELATED APPLICATION

The present application is a national stage application of International Patent Application No. PCT/CN2021/135273, filed on Dec. 3, 2021, which claims priority to the Chinese Patent Application No. 2020115542198, filed with the China National Intellectual Property Administration (CNIPA) on Dec. 24, 2020, and entitled “MOBILE PHONE-BASED BIOLOGICAL TESTING METHOD AND APPARATUS”, which is incorporated herein by reference in its entirety.

### TECHNICAL FIELD

The present disclosure relates to the technical field of biological testing, and in particular, to a mobile phone-based biological testing method and apparatus.

### BACKGROUND

With the rapid development, the point-of-care testing (POCT) technology can be used to quantitatively and qualitatively detect multiple biomarkers, so as to help assess people's health. But so far, all quantitative POCT technologies require a “reader” that quantifies a reaction signal (such as fluorescence). The commonly used “reader” includes at least two parts. One is the excitation part, that is, to excite (irradiate) the signal substance in the tested object with a light beam of a certain wavelength. The other is the receiving part, which receives and quantifies the signal emitted by the signal substance after it is excited (irradiated). The fluorescence reader is such a typical instrument. Such readers, which contain the necessary electronics and optical paths, are limited in size and cost and are usually used in medical institutions, not in families and by individuals. Obviously, the ordinary individuals do not buy a “reader” for testing one or two items. Some POCT technologies, such as colloidal gold immunochromatography assay (GICA), can be read with the naked eye without a “reader”. However, due to the difficulty in controlling the inter-batch difference and the narrow dynamic range of signals, the GICA is not quantitative, and can only qualitatively determine the “presence” or “absence” of the tested object, such as the human chorionic gonadotropin (HCG) pregnancy test strip.

In addition, now almost all the POCT products can only be subjected to single inspection, that is, only one biomarker can be tested for one sample at a time. The reason is that the signals of multiple biomarkers need to be distinguished either spatially or spectroscopically. Simple spatial separation (such as multiple-detection card) is bound to increase the volumes of the reagent card and the sample. If the signal of each biomarker is resolved from the spectrum, multiple light splitters and filters need to be added to the optical path, which is complicated and costly.

Moreover, existing quantitative readers are faced with the challenge of dynamic range, that is, the obtained signal intensity is only proportional with the concentration of the tested object in a certain range. Above this range, the signal intensity tends to be saturated and no longer increases. Under this range, the signal intensity tends to be zero or a certain value and does not decrease. This challenge is particularly obvious for POCT readers, because they are often limited in size and cost. Therefore, the measurement dynamic range of the POCT readers generally can only reach about two orders of magnitude.

### SUMMARY

An objective of the present disclosure is to provide a mobile phone-based biological testing method and apparatus, which can greatly extend functions of a mobile phone and achieve multiplex quantitative testing in a wide dynamic range.

To achieve the above objective, the present disclosure provides the following solutions:

A mobile phone-based biological testing apparatus includes: a mobile phone and an ultraviolet (UV) light-emitting diode (LED) light source arranged on the mobile phone.

The UV LED light source is configured to emit excitation light, and the excitation light excites a to-be-tested sample.

The mobile phone is configured to photograph an obtained excited to-be-tested sample, so as to obtain an original image file. The original image file is in a RAW format with a 16-bit depth.

The mobile phone is further configured to process the original image file to obtain a concentration of the to-be-tested sample.

Optionally, the excitation light is invisible light.

Optionally, the mobile phone-based biological testing apparatus further includes: a lens arranged on an outgoing optical path of the UV LED light source and configured to make the excitation light uniformly and intensively irradiate the to-be-tested sample.

Optionally, the lens is a Fresnel lens.

Optionally, the mobile phone-based biological testing apparatus further includes: a long-pass filter arranged in front of a camera of the mobile phone and configured to filter the excitation light.

Optionally, the mobile phone-based biological testing apparatus further includes: a fluorescent immunochromatographic test strip configured to place the to-be-tested sample and including multiple control lines.

Optionally, the mobile phone-based biological testing apparatus further includes: a cassette. The fluorescent immunochromatographic test strip is placed in the cassette; and the mobile phone is placed in or on the cassette.

A mobile phone-based biological testing method is provided, applied to the above mobile phone-based biological testing apparatus, and including:
according to a spectral response curve of a complementary metal oxide semiconductor (CMOS) photosensitive unit of the mobile phone, selecting N kinds of signal substances emitting visible light of specific wavelengths excited by UV light to label N to-be-tested biomarkers in the to-be-tested sample, so as to obtain to-be-tested biomarker-signal substance compounds, where N is 1, 2 or 3;
exciting the to-be-tested biomarker-signal substance compounds using the excitation light;
photographing obtained excited to-be-tested biomarker-signal substance compounds for multiple times based on combinations of different photosensitivities and different exposure times, so as to obtain a first original image file in a RAW format with a 16-bit depth;
photographing unlabeled signal substances to obtain a second original image file in a RAW format with a 16-bit depth;
calculating a matrix of transition coefficients of fluorescence intensities of the signal substances received by channels according to red, green and blue (RGB) or red, yellow and blue (RYB) channel data of a pixel in the second original image file;
calculating a fluorescence intensity of each of the to-be-tested biomarker-signal substance compounds according to the matrix of transition coefficients and RGB or RYB channel data of a pixel in the first original image file; and
calculating a concentration of each of the to-be-tested biomarkers according to the fluorescence intensity, so as to complete biological testing.

Optionally, a process of calculating the concentration of each of the to-be-tested biomarkers according to the fluorescence intensity specifically includes:
interpolating a calibration curve using the fluorescence intensity to obtain the concentration of each of the to-be-tested biomarkers, where the calibration curve is established according to fluorescence intensities of calibrator-signal substance compounds of different concentrations.

Optionally, the signal substances are quantum dots or time-resolved fluorescent microspheres.

According to specific examples provided by the present disclosure, the present disclosure discloses the following technical effects:

The present disclosure discloses a mobile phone-based biological testing method and apparatus. The method includes: according to a spectral response curve of a CMOS photosensitive unit of the mobile phone, selecting N kinds of signal substances emitting visible light of specific wavelengths excited by UV light to label N to-be-tested biomarkers in the to-be-tested sample, so as to obtain to-be-tested biomarker-signal substance compounds, where N is 1, 2 or 3; exciting the to-be-tested biomarker-signal substance compounds using the excitation light; photographing obtained excited to-be-tested biomarker-signal substance compounds for multiple times based on combinations of different photosensitivities and different exposure times, so as to obtain a first original image file in a RAW format with a 16-bit depth; photographing unlabeled signal substances to obtain a second original image file in a RAW format with a 16-bit depth; calculating a matrix of transition coefficients of fluorescence intensities of the signal substances received by channels according to RGB or RYB channel data of a pixel in the second original image file; calculating a fluorescence intensity of each of the to-be-tested biomarker-signal substance compounds according to the matrix of transition coefficients and RGB or RYB channel data of a pixel in the first original image file; and calculating the concentration of each of the to-be-tested biomarkers according to the fluorescence intensity, so as to complete biological testing. The present disclosure greatly extends functions of a mobile phone, and can achieve multiplex quantitative biological testing in a wide dynamic range.

### BRIEF DESCRIPTION OF THE DRAWINGS

To describe the technical solutions in the examples of the present disclosure or in the prior art more clearly, the accompanying drawings required for the examples are briefly described below. Apparently, the accompanying drawings in the following description show merely some examples of the present disclosure, and those of ordinary skill in the art may still derive other accompanying drawings from these accompanying drawings without creative efforts.

FIG. 1 is a structural diagram of a mobile phone-based biological testing apparatus provided by an example of the present disclosure;

FIG. 2 is a schematic diagram of CMOS spectral response curves and wavelengths of quantum dots provided by the example of the present disclosure;

FIG. 3 is a schematic diagram of a fluorescent immunochromatographic test strip provided by the example of the present disclosure;

FIG. 4 is a calibration curve graph provided by the example of the present disclosure;

FIG. 5 is a schematic diagram I of a concentration testing range provided by the example of the present disclosure;

FIG. 6 is a schematic diagram II of a concentration testing range provided by the example of the present disclosure;

FIG. 7 is a schematic diagram of a CMOS filter array of a mobile phone provided by the example of the present disclosure;

FIG. 8 is a schematic diagram of CMOS photosensitive units provided by the example of the present disclosure; and

FIG. 9 is a schematic diagram of a color pixel composed of the CMOS photosensitive units provided by the example of the present disclosure.

### REFERENCE NUMERALS

1—UV LED light source, 2—lens, 3—long-pass filter, 41—blue quantum dot, 42—green quantum dot, 43—red quantum dot, 5—lens of mobile phone, 6—first C line, 7—second C line, 11—anti-mullerian hormone (AMH) antibody A2, 12—anti-luteinizing hormone (LH) antibody B2, 13—anti-follicle stimulating hormone (FSH) antibody C2, 21—AMH antibody A1, 22—anti-LH antibody B1, and 23—anti-FSH antibody C1.

### DETAILED DESCRIPTION OF THE EMBODIMENTS

The technical solutions of the examples of the present disclosure are clearly and completely described below with reference to the accompanying drawings. Apparently, the described examples are merely a part rather than all of the examples of the present disclosure. All other examples obtained by those of ordinary skill in the art based on the examples of the present disclosure without creative efforts shall fall within the protection scope of the present disclosure.

The mobile phone has powerful functions. The present disclosure ingeniously utilizes the photoelectric principle of color photography of the existing mobile phone and its components, and minimally adds appropriate devices to it, transforming it into a convenient biological testing platform with wide dynamic range and multiplex quantitative characteristics.

An objective of the present disclosure is to provide a mobile phone-based biological testing method and apparatus, which can greatly extend functions of a mobile phone and achieve multiplex quantitative testing in a wide dynamic range.

To make the above objectives, features, and advantages of the present disclosure clearer and more comprehensible, the present disclosure will be further described in detail below with reference to the accompanying drawings and the specific examples.

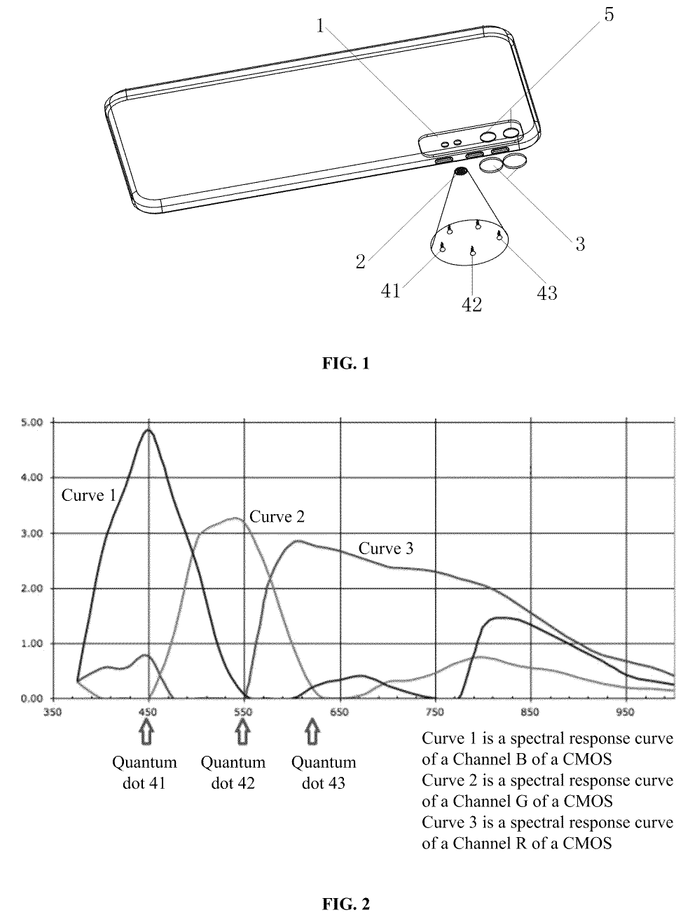

FIG. 1 is a structural diagram of a mobile phone-based biological testing apparatus provided by an example of the present disclosure. As shown in FIG. 1, the mobile phone-based biological testing apparatus includes: a mobile phone and a UV LED light source 1 arranged on the mobile phone. The UV LED light source 1 was configured to emit excitation light, and the excitation light excited a to-be-tested sample. The mobile phone was configured to photograph an obtained excited to-be-tested sample, so as to obtain an original image file. The original image file was in a RAW format with a 16-bit depth. The mobile phone was further configured to process the original image file to obtain a concentration of the to-be-tested sample. The excitation light emitted by the UV LED light source 1 was invisible light.

In this example, the mobile phone-based biological testing apparatus further included: a lens 2, a long-pass filter 3, a fluorescent immunochromatographic test strip, and a cassette.

The lens 2 was arranged on an outgoing optical path of the UV LED light source 1 and configured to make the excitation light uniformly and intensively irradiate the to-be-tested sample. The lens 2 was a Fresnel lens.

The long-pass filter 3 was arranged in front of a camera of the mobile phone and configured to filter the excitation light.

The fluorescent immunochromatographic test strip was configured to place the to-be-tested sample and included multiple control lines.

The fluorescent immunochromatographic test strip was placed in the cassette. The mobile phone was placed in or on the cassette.

This example further provided a mobile phone-based biological testing method, applied to the above mobile phone-based biological testing apparatus, and including the following steps.

According to a spectral response curve of a CMOS photosensitive unit of the mobile phone, N kinds of signal substances emitting visible light of specific wavelengths excited by UV light were selected to label N to-be-tested biomarkers in the to-be-tested sample, so as to obtain to-be-tested biomarker-signal substance compounds, where N was 1, 2 or 3.

The to-be-tested biomarker-signal substance compounds were excited using the excitation light.

Obtained excited to-be-tested biomarker-signal substance compounds were photographed for multiple times based on combinations of different photosensitivities and different exposure times, so as to obtain a first original image file in a RAW format with a 16-bit depth.

Unlabeled signal substances were photographed to obtain a second original image file in a RAW format with a 16-bit depth.

A matrix of transition coefficients of fluorescence intensities of the signal substances received by channels was calculated according to RGB or RYB channel data of a pixel in the second original image file.

A fluorescence intensity of each of the to-be-tested biomarker-signal substance compounds was calculated according to the matrix of transition coefficients and RGB or RYB channel data of a pixel in the first original image file.

A concentration of each of the to-be-tested biomarkers was calculated according to the fluorescence intensity, so as to complete biological testing.

A calibration curve was interpolated using the fluorescence intensity to obtain the concentration of each of the to-be-tested biomarkers. The calibration curve was established according to fluorescence intensities of calibrator-signal substance compounds of different concentrations.

The signal substances were quantum dots or time-resolved fluorescent microspheres.

The principle of the present disclosure was described in detail below.

1. Mobile Phone Hardware

Excitation Part:

As shown in FIG. 1, a UV LED light source 1 was built in an existing mobile phone in the present disclosure as the excitation part. The built-in UV LED light source 1 was a patch LED that could be placed together or separated from other light sources on the back of the mobile phone. The specific parameters were as follows.

In order to minimize the use of mobile phone space, each side of the UV LED light source 1 was less than 4 mm.

In order to obtain sufficient luminous flux, the UV LED light source 1 had chip power of 0.2-3 W, and a central wavelength of the chip was less than 400 nm, preferably 365 nm, 340 nm or 290 nm.

The UV LED light source 1 did not emit visible light.

In order to make the UV LED light source 1 uniform and relatively concentrated in the tested area (about 5 cm×5 cm), a lens 2 was placed in front of the UV LED light source 1 (for clarity, in FIG. 1, the lens 2 and the UV LED light source 1 were separated by an appropriate distance in a direction perpendicular to the screen of the mobile phone). The parameters of the lens 2 were as follows:
Divergence angle: 30-60°.
UV light transmittance: >80%.
Focal length: 10-25 cm.

Receiving Part:

In the present disclosure, a long-pass filter 3 was added in front of a camera of the mobile phone as the receiving part (for clarity, in FIG. 1, the long-pass filter 3 and the lens of the mobile phone 5 were separated by an appropriate distance in a direction perpendicular to the screen of the mobile phone). The parameters of the long-pass filter 3 were as follows:
Central wavelength: 400 nm+/−5 nm
Cut-off depth: OD 3.0-4.0
Transmittance: >95%
Incidence angle: 0°

In this way, the UV excitation light was cut off, and most of the visible light could pass through the long-pass filter 3, without any impact on the existing picture taking or photography of the mobile phone. In addition, in order to obtain approximately vertical excitation and reception, the distance between the receiving part and the excitation part in the present disclosure was within 2 cm.

2. Mobile Phone Photographing Detection and Control Process and Wide Dynamic and Multiplex Quantitative Testing Process

In this example, the APP was responsible for controlling the photography of the mobile phone, and the APP had the permission to retrieve the “professional” mode of the photography of the mobile phone and store the obtained image as an RAW file. A process is as follows.

(1) The user started the APP.

(2) The APP scanned the identification code of reagent cards (test strip, chip, etc.) used for testing to identify the specific testing item (under a testing item, multiple biomarkers could be tested at the same time).

(3) The APP prompted the user to confirm the item.

(4) The APP automatically identified the ambient light intensity and prompted the user to enter an environment with low light intensity.

(5) If the ambient light intensity met the requirements for photography, the user was guided and prompted to place the testing reagent card (test strip, chip, etc.) at the nearest clear imaging distance, that is, 5-10 cm, directly below the UV LED light source of the mobile phone.

(6) The stored photographing parameters were retrieved. The UV LED light source was switched on. The testing reagent card (test strip, etc.) was photographed for multiple times with different photographing hardware parameters (such as combinations of exposure time and photosensitivities (ISO)) to obtain the image, and the image was stored in the RAW format.

In order to achieve a wide dynamic range of testing, for to-be-tested biomarkers of a low concentration, the image was obtained with long exposure time (up to 1-2 s) and high photosensitivity (up to ISO800-1600). For to-be-tested biomarkers of a high concentration, the image was obtained with short exposure time (as short as 1/200 s) and low photosensitivity (ISO50). Finally, according to the signal intensity of the calibrator under the specific photography hardware parameters, the signal intensities and concentrations of the to-be-tested biomarkers under the same conditions were calculated. In the process of implementation, the testing items were known and the reagent cards (test strips, chips, etc.) had been prepared. Therefore, for each item and each reagent card (test strip, chip, etc.), the present disclosure pre-stored hardware parameters (ISO value and exposure time, etc.) that had been optimized for the required dynamic range, and only need to identify the testing item through the barcode on the reagent card (test strip, chip, etc.) during the test, so as to retrieve the corresponding stored photography hardware parameters to achieve a wide dynamic range of testing for each item.

(7) In the case of simultaneous testing of multiple biomarkers (multiplex testing), the response spectral curves of three channels (RGB channel or RYB channel, and the RGB channel was taken as an example in this example) of the photosensitive CMOS of the camera of the mobile phone were used to select three quantum dots of specific emission wavelengths as signal substances to label the to-be-tested biomarkers. The emission wavelengths of the three quantum dots corresponded to the spectral response peaks of the three RGB channels, and were at the lowest value of the spectral response of the other channels. FIG. 2 showed the spectral response curve of a single photosensitive unit in three channels of the CMOS of a mobile phone. Blue quantum dots 41 with a peak emission wavelength of about 450 nm, green quantum dots 42 with a peak emission wavelength of about 550 nm and red quantum dots 43 with a peak emission wavelength of about 630 nm were selected as the labeled signal substances. It shall be emphasized that any fluorescent substance can be used as a signal substance as long as it emits visible light of a specific wavelength excited by UV light. In this example, the quantum dot was taken as an example.

The mobile phone photographed a mixture of the three to-be-tested biomarker-quantum dot compounds, and obtained the reading of the RGB channel of each pixel from the RAW file. The fluorescence intensity of each signal substance could be obtained by a conversion matrix M:

$$[ \begin{matrix}{S}_{1} \\ {S}_{2} \\ {S}_{3}\end{matrix} ] = M [ \begin{matrix}B \\ G \\ R\end{matrix} ] ,$$

where B, G and R were fluorescence readings by channel B, channel G and channel R respectively, S1, S2 and S3 were fluorescence intensities emitted by the quantum dots 41, 42 and 43, and the matrix M was a conversion matrix. The process of obtaining M is as follows.

It was not difficult to see that for any photosensitive unit:

$$\{ \begin{matrix}B = {a}_{11} {S}_{1} + {a}_{12} {S}_{2} + {a}_{13} {S}_{3} \\ G = {a}_{21} {S}_{1} + {a}_{22} {S}_{2} + {a}_{23} {S}_{3} \\ R = {a}_{31} {S}_{1} + {a}_{32} {S}_{2} + {a}_{33} {S}_{3}\end{matrix} ,$$

namely:

$$[ \begin{matrix}B \\ G \\ R\end{matrix} ] = A [ \begin{matrix}{S}_{1} \\ {S}_{2} \\ {S}_{3}\end{matrix} ] ,$$

where A was a matrix of transition coefficients of fluorescence intensities of each quantum dot received by each channel:

$$A = [ \begin{matrix}{a}_{11} & {a}_{12} & {a}_{13} \\ {a}_{21} & {a}_{22} & {a}_{23} \\ {a}_{31} & {a}_{32} & {a}_{33}\end{matrix} ] .$$

Diagonal elements of the matrix A were much greater than the rest. Each element of the matrix A was obtained by photographing a single signal substance as follows. That is, the quantum dots 41 were photographed first, a reading a₁₁ was obtained from the channel B, a reading a₂₁ was obtained from the channel G, a reading a₃₁ was obtained from the channel R, and the readings were normalized. The quantum dots 42 were photographed, a reading a₁₂ was obtained from the channel B, a reading a₂₂ was obtained from the channel G, a reading a₃₂ was obtained from the channel R, and the readings were normalized. Finally, the quantum dots 43 were photographed, a reading a₁₃ was obtained from the channel B, a reading a₂₃ was obtained from the channel G, a reading a₃₃ was obtained from the channel R, and the readings were normalized.

After the matrix A was obtained, the matrix M was the inverse of matrix A, and each element of the matrix M was obtained after taking the inverse of A, M=A⁻¹.

It shall be emphasized that the data used for the multiplex quantitative testing and analysis of the present disclosure was the direct reading of each photosensitive unit of the mobile phone, that is, the data of the RAW file, rather than the RGB value of each pixel of the JPEG image file that was usually calibrated by some algorithm and correction for display.

After the fluorescence intensities S1, S2 and S3 of each signal substance were obtained, the calibration curves of the to-be-tested biomarkers under the same photography hardware parameters were retrieved from the APP and used for interpolation, that is S1 was interpolated into the calibration curve 1, S2 was interpolated into the calibration curve 2, and S3 was interpolated into the calibration curve 3. Finally, the concentration of the test biomarker corresponding to each signal substance was obtained.

The process of obtaining the calibration curves 1-3 was as follows.

According to the above process, the calibrators (calibrator-quantum dot compounds) of the test biomarker 1 of different concentrations labeled by the signal substance 1 were photographed and converted to obtain the signal intensities S_{1A}, S_{1B}, S_{1C} . . . S_{1N} of the calibrators 1A, 1B, 1C . . . 1N, and the calibration curve was further made. The calibrators of the test biomarker 2 of different concentrations labeled by the signal substance 2 were photographed and converted to obtain the signal intensities S_{2A}, S_{2B}, S_{2C} . . . S_{2N} of the calibrators 2A, 2B, 2C . . . 2N, and the calibration curve 2 was further made. The calibrators of the test biomarker 3 of different concentrations labeled by the signal substance 3 were photographed and converted to obtain the signal intensities S_{3A}, S_{3B}, S_{3C} . . . S_{3N} of the calibrators 3A, 3B, 3C . . . 3N, and the calibration curve 3 was further made. These calibration curves were then pre-stored in the APP.

(8) According to the concentration of each test biomarker, combined with the clinical medical implication of the project, the results were analyzed and interpreted, and a report was issued to the user.

3. Immunochromatographic Test Strip (Reagent Card)

The immunochromatographic test strip was one of the most commonly used methods of POCT. When the fluorescent immunochromatographic test strip was used for quantification, the concentration of the tested substance was usually measured using the method of dividing the T line (test line) signal by the C line (control line) signal (T/C).

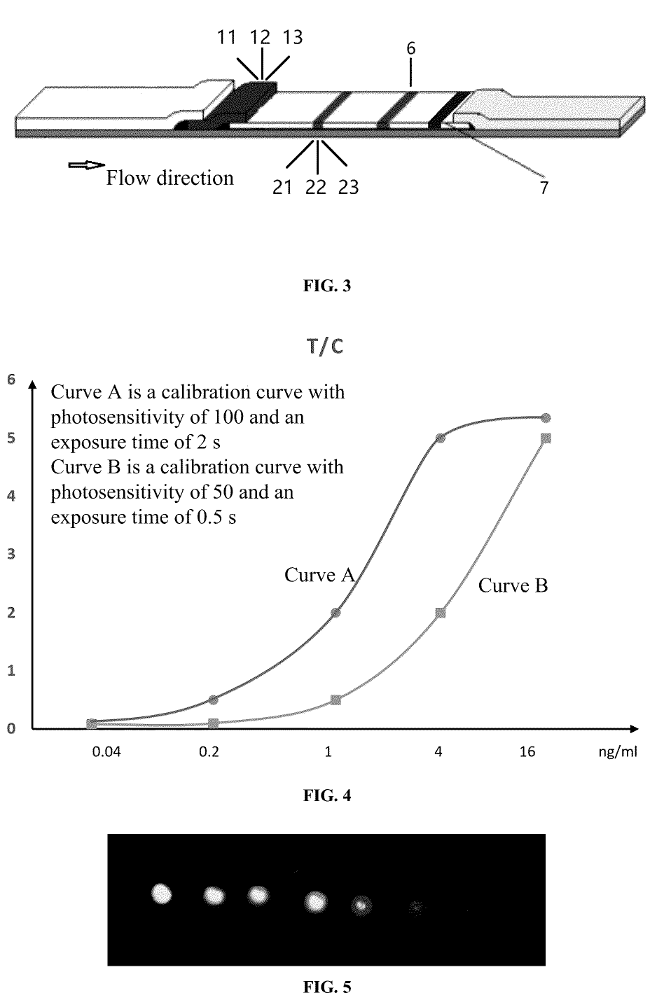

In order to achieve a wide dynamic range of testing, combined with combinations of different exposure times and ISO values (photosensitivities), the present disclosure adopted two control lines. FIG. 3 shows a first C line 6 and a second C line 7. The first C line 6 was weak, and the second C line 7 was strong. At long exposure times and high ISO, T/C was calculated with the first C line 6. At short exposure times and low ISO, T/C was calculated using the second C line 7. In a specific example, the present disclosure labeled the AMH antibody with 630 nm quantum dots. Two C lines were used to test the AMH standard. As shown in FIG. 4, an Android phone was used. In this figure, curve A was the calibration curve when the photosensitivity was 100 and the exposure time was 2 s, and curve B is the calibration curve when the photosensitivity was 50 and the exposure time was 0.5 s. Through combination, the present disclosure could obtain a dynamic testing range of 0.04-16 ng/mL of AMH, which was greater than two orders of magnitude and superior to the dynamic testing range of most POCT products using fluorescence readers.

In the implementation of multiplex testing, different from the ordinary multi-biomarker (or multiplex) immunochromatographic test strip, the immunochromatographic test strip of the present disclosure required only one T line, which contained antigens or antibodies of multiple test biomarkers.

In a specific example, the present disclosure can simultaneously test hormones AMH, LH and FSH in female blood using the fluorescent immunochromatographic test strip. All three hormones were protein molecules and could be tested using the sandwich method.

(1) The T line of the present disclosure was marked with antibodies to these three biomarkers simultaneously, that is, the T line was marked with a mixture of the AMH antibody A1 (21 in FIG. 3), the anti-LH antibody B1 (22 in FIG. 3) and the anti-FSH antibody C1 (23 in FIG. 3).

(2) The conjugation pad of the present disclosure contained the AMH antibody A2 (11 in FIG. 3) labeled by red quantum dots, the anti-LH antibody B2 (12 in FIG. 3) labeled by blue quantum dots, and the anti-FSH antibody C2 (13 in FIG. 3) labeled by green quantum dots. The present disclosure might also have three layers of conjugation pads, which were overlapped in sequence. Each layer respectively contained the AMH antibody A2 (11 in FIG. 3) labeled by red quantum dots, the anti-LH antibody B2 (12 in FIG. 3) labeled by blue quantum dots, and the anti-FSH antibody C2 (13 in FIG. 3) labeled by green quantum dots.

It could be understood that fluorescent immunochromatographic test strip was optional in the present disclosure, and the biological testing in the present disclosure could also be in the form of other test strips, reagent cards or chips.

4. Cassette

In the dark environment, such as the indoor environment that was dark at night, the visible light in the environment was low enough to not cause any interference to the light emitted by the quantum dot, and the present disclosure could directly use the mobile phone for quantification.

In the daytime environment, as an option, the present disclosure used a cassette to ensure that the position of the mobile phone relative to the testing reagent card was fixed and the whole optical path was not affected by the ambient light. Because the intensity and direction of the ambient light were difficult to control, in order to obtain a uniform field of view and not be interfered with by the environment, the present disclosure could be configured with a cassette for the mobile phone, and placed the camera of the mobile phone and the UV LED light source 1 on the top of the cassette, or arranged the mobile phone in the cassette, placing the reagent card at the bottom of the cassette, and aligning the camera and the UV LED light source 1 with the reagent card. In the whole process of photographing detection, a uniform lighting environment could be obtained. The mobile phone and the reagent card were at an optimal distance to obtain a clear image. The position of the mobile phone and the reagent card were fixed to prevent hand-held shaking and to take stable images with long exposures. The entire cassette did not contain any photoelectric devices.

For convenience of carrying, the cassette in the present disclosure can be a foldable cassette.

This example further provided a specific implementation, which was as follows.

In order to obtain a wide dynamic range of testing, the present disclosure used fluorescent quantum dots as signal substances. Compared with other fluorescent molecules, quantum dots were excited by UV light to emit visible light. The distance between the two wavelengths (Stokes shift) was large, which makes optical path design and signal detection easy. In addition, the quantum dot had excellent optical stability withstanding long time irradiation of excitation light source without obvious optical performance loss, and was suitable for long time exposure and multiple exposure of the present disclosure. In addition, under the same excitation intensity, the quantum dots had high fluorescence intensity, which was useful for the realization of a wide dynamic range. The fluorescence intensity of quantum dots was 20 times higher than that of “Rhodamine 6G”, the most commonly used organic fluorescent material, and its stability was at least 100 times higher than that of “Rhodamine 6G”.

In order to obtain a wide dynamic range of testing, the present disclosure adopted the RAW format to store image files instead of the JPEG files for display. The RAW file was the original data recorded by each photosensitive unit of the CMOS of the mobile phone, which directly reflected the optical signal received by each photosensitive unit. In addition, the file storage bit depth of the RAW format was 16 bits, more than 8 bits of the JPEG format.

In order to obtain a wide dynamic range of testing, the present disclosure used a high dynamic range (HDR) algorithm for mobile phone photography. The CMOS photosensitive units commonly used in the mobile phones had limited dynamic range. Under certain photographing conditions (such as exposure time and ISO), the signal that was too strong was represented as saturation, while the signal that was too weak was represented as 0 or a low value (device noise). In order to achieve a wider dynamic range of quantitative testing, the present disclosure used combinations of different exposure times and ISO values to photograph reagent cards (such as fluorescent immunochromatographic test strips) for multiple times to obtain images, and used different images to quantify different spots.

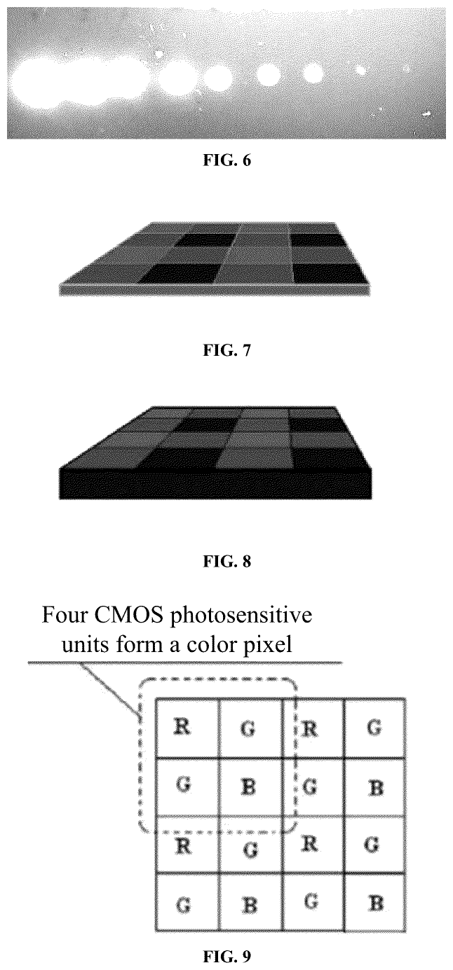

As shown in FIG. 5 to FIG. 6, an Android phone was used to photograph quantum dot spots of different concentrations on the nitrocellulose membrane with the UV light source (365 nm, 3 W). Each quantum dot spot had a volume of 0.5 ul and a concentration 1/2.5 of the previous quantum dot spot.

The photography parameters in FIG. 5 were:
ISO: 50.
Exposure time: 0.5 s.
Storage format: RAW.
The photography parameters in FIG. 6 are:
ISO: 100.
Exposure time: 5 s.
Storage format: RAW.

It can be seen that in FIG. 5, the present disclosure can detect the 1-5 spots, and in FIG. 6, the present disclosure can detect the 4-9 spots. Through combination, the present disclosure can obtain a dynamic testing range of 2.5⁸=1.5×10³ times for quantum dots, more than three orders of magnitude. The dynamic testing range was comparable to that of most professional immunofluorescence readers.

In the process of implementation, the testing items were known and the reagent cards (test strips, chips, etc.) were prepared. Therefore, for each item and each reagent card (test strip, chip, etc.), the present disclosure pre-stored hardware parameters (ISO value and exposure time, etc.) that had been optimized for the required dynamic range. During the test, the testing item could be known through the barcode on the reagent card (test strip, chip, etc.), so as to retrieve the corresponding stored photography hardware parameters to achieve a wide dynamic range of testing for each item.

The most commonly used working principle of color CMOS for mobile phone lenses was color single CMOS, that is, a CMOS array was covered with RGB three-color filters, usually in a ratio of 1:2:1 (as shown in FIG. 7 to FIG. 8), and four photosensitive units formed a color pixel (that is, the red and blue filters each covered one photosensitive unit, and the remaining two photosensitive units were covered with the green filters, as shown in FIG. 9). In order to obtain similar images to those perceived by human eyes, after CMOS output the RAW files, a series of algorithms and calibration were usually required to recover the RGB signals of each unit of this group of photosensitive units, store them in files, and display them on the screen of a computer or a mobile phone. However, as quantitative testing, what was more concerned about was the light intensity of each signal substance, rather than the “true color” of the image presented after restoration to the human eye.

The present disclosure ingeniously selected quantum dots of three colors as signal substances by utilizing the spectral response curve of the RGB photosensitive unit in the photosensitive CMOS of the camera, and made full use of the existing optical devices of the mobile phone and the output of the RAW file. Therefore, which color of signal substances the light intensity came from could be known without additional filters, thus realizing simultaneous testing of multiple biomarkers, that is, multiplex testing.

As shown in FIG. 2, in a case of multiplex testing, the present disclosure selected blue quantum dots 41 with a peak emission wavelength of about 450 nm, green quantum dots 42 with a peak emission wavelength of about 550 nm and red quantum dots 43 with a peak emission wavelength of about 630 nm as the labeled signal substances. On one hand, the wavelengths of the three colors were relatively separated and did not interfere with each other. On the other hand, the wavelengths of the three colors corresponded to the peak of the B, G, and R channel in the photosensitive CMOS of the camera of the mobile phone, and were at the lowest values of the responses of the other channels (arrow in FIG. 2).

An Android phone (exposure time of 1/100 s, ISO of 200) was used to photograph the quantum dots 41 (100 nM). A reading a₁₁ was obtained from the channel B, a reading a₂₁ was obtained from the channel G, a reading a₃₁ was obtained from the channel R, and the readings were normalized (for example, dividing them by a₁₁). The quantum dots 42 (100 nM) were photographed, a reading a₁₂ was obtained from the channel B, a reading a₂₂ was obtained from the channel G, a reading a₃₂ was obtained from the channel R, and the readings were normalized (for example, dividing them by a₂₂). The quantum dot 43 (100 nM) were photographed, a reading a₁₃ was obtained from the channel B, a reading a₂₃ was obtained from the channel G, a reading a₃₃ was obtained from the channel R, and the readings were normalized (for example, dividing them by a₃₃). Therefore, the coefficient matrix A was obtained:

$$A = [ \begin{matrix}1 & 0.121 & 0.066 \\ 0.2538 & 1 & 0.283 \\ 0.0575 & 0.118 & 1\end{matrix} ] ,$$

such that the conversion matrix M was obtained:

$$M = {A}^{- 1} = [ \begin{matrix}1.0326 & - 0.1209 & - 0.0339 \\ - 0.2538 & 1.0643 & - 0.2844 \\ - 0.0294 & - 0.1186 & 1.0355\end{matrix} ] .$$

The principle and experimental method of this example could also be applied to RYB filter arrays.

According to specific examples provided by the present disclosure, the present disclosure discloses the following technical effects:

1. The present disclosure greatly extends functions of a mobile phone and makes it become a mobile platform for personal health test. Many items (such as AMH test and urinary micro-albumin test) that individuals can acquire samples (such as fingertip blood, urine and saliva) can be quantitatively tested using a mobile phone.

2. The present disclosure adjusts the hardware parameters of mobile phone photography and uses the output of multiple RAW files as original data for quantitative test, and the dynamic range can be more than two orders of magnitude. For example, the present disclosure is used to measure the AMH in the fingertip blood of female, and the detection range is 0.04-16 ng/mL, thus making it possible to accurately predict the age of female menopause and evaluate the function of female ovarian reserve.

3. The present disclosure adopts fluorescent quantum dots with special emission spectrum and special fluorescent immunochromatographic test strip design, so as to conduct quantitative test of multiple biomarkers at the same time. This is extremely useful for some scenarios, for example, in the present disclosure, only 80 μl of fingertip blood needs to be collected for multiplex testing of AMH, FSH and LH of female simultaneously, so as to conduct a comprehensive assessment of the ovarian function of female.

4. The present disclosure provides an algorithm for converting the RGB channel reading in the RAW file into fluorescence intensity of each signal substance, and experimental steps for obtaining each element of the conversion matrix in the algorithm. The conversion matrix has only 9 elements, and each element can be obtained in advance, so the algorithm has the characteristics of simple conversion and fast implementation.

5. The present disclosure has very low hardware requirements for the mobile phone, and does not need to change the existing optical path of the mobile phone, and only an UV LED light source, a condenser lens and a filter need to be added, which is extremely cost-effective.

Each example of the present specification is described in a progressive manner, each example focuses on the difference from other examples, and the same and similar parts between the examples may refer to each other.

Specific examples are used herein to explain the principles and implementations of the present disclosure. The foregoing description of the examples is merely intended to help understand the method of the present disclosure and its core ideas; besides, various modifications may be made by those of ordinary skill in the art to specific implementations and the scope of application in accordance with the ideas of the present disclosure. In conclusion, the content of the present specification shall not be construed as limitations to the present disclosure.

1.-10. (canceled)

11. A mobile phone-based biological testing apparatus, comprising a mobile phone and an ultraviolet (UV) light-emitting diode (LED) light source arranged on the mobile phone, wherein
the UV LED light source is configured to emit excitation light, and the excitation light excites a to-be-tested sample;
the mobile phone is configured to photograph an obtained excited to-be-tested sample, so as to obtain an original image file, wherein the original image file is in a RAW format with a 16-bit depth; and
the mobile phone is further configured to process the original image file to obtain a concentration of the to-be-tested sample.

12. The mobile phone-based biological testing apparatus according to claim 11, wherein the excitation light is invisible light.

13. The mobile phone-based biological testing apparatus according to claim 11, further comprising: a lens arranged on an outgoing optical path of the UV LED light source and configured to make the excitation light uniformly and intensively irradiate the to-be-tested sample.

14. The mobile phone-based biological testing apparatus according to claim 13, wherein the lens is a Fresnel lens.

15. The mobile phone-based biological testing apparatus according to claim 11, further comprising: a long-pass filter arranged in front of a camera of the mobile phone and configured to filter the excitation light.

16. The mobile phone-based biological testing apparatus according to claim 11, further comprising: a fluorescent immunochromatographic test strip configured to place the to-be-tested sample and comprising multiple control lines.

17. The mobile phone-based biological testing apparatus according to claim 16, further comprising: a cassette, wherein the fluorescent immunochromatographic test strip is placed in the cassette; and the mobile phone is placed in or on the cassette.

18. A mobile phone-based biological testing method, applied to the mobile phone-based biological testing apparatus according to claim 1, and comprising:
according to a spectral response curve of a complementary metal oxide semiconductor (CMOS) photosensitive unit of the mobile phone, selecting N kinds of signal substances emitting visible light of specific wavelengths excited by UV light to label N to-be-tested biomarkers in the to-be-tested sample, so as to obtain to-be-tested biomarker-signal substance compounds, wherein N is 1, 2 or 3;
exciting the to-be-tested biomarker-signal substance compounds using the excitation light;
photographing obtained excited to-be-tested biomarker-signal substance compounds for multiple times based on combinations of different photosensitivities and different exposure times, so as to obtain a first original image file in a RAW format with a 16-bit depth;
photographing unlabeled signal substances to obtain a second original image file in a RAW format with a 16-bit depth;
calculating a matrix of transition coefficients of fluorescence intensities of the signal substances received by channels according to red, green and blue (RGB) or red, yellow and blue (RYB) channel data of a pixel in the second original image file;
calculating a fluorescence intensity of each of the to-be-tested biomarker-signal substance compounds according to the matrix of transition coefficients and RGB or RYB channel data of a pixel in the first original image file; and
calculating a concentration of each of the to-be-tested biomarkers according to the fluorescence intensity, so as to complete biological testing.

19. The mobile phone-based biological testing method according to claim 18, wherein the excitation light is invisible light.

20. The mobile phone-based biological testing method according to claim 19, wherein a process of calculating the concentration of each of the to-be-tested biomarkers according to the fluorescence intensity specifically comprises:
interpolating a calibration curve using the fluorescence intensity to obtain the concentration of each of the to-be-tested biomarkers, wherein the calibration curve is established according to fluorescence intensities of calibrator-signal substance compounds of different concentrations.

21. The mobile phone-based biological testing method according to claim 18, further comprising: a lens arranged on an outgoing optical path of the UV LED light source and configured to make the excitation light uniformly and intensively irradiate the to-be-tested sample.

22. The mobile phone-based biological testing method according to claim 21, wherein a process of calculating the concentration of each of the to-be-tested biomarkers according to the fluorescence intensity specifically comprises:
interpolating a calibration curve using the fluorescence intensity to obtain the concentration of each of the to-be-tested biomarkers, wherein the calibration curve is established according to fluorescence intensities of calibrator-signal substance compounds of different concentrations.

23. The mobile phone-based biological testing method according to claim 21, wherein the lens is a Fresnel lens.

24. The mobile phone-based biological testing method according to claim 23, wherein a process of calculating the concentration of each of the to-be-tested biomarkers according to the fluorescence intensity specifically comprises:
interpolating a calibration curve using the fluorescence intensity to obtain the concentration of each of the to-be-tested biomarkers, wherein the calibration curve is established according to fluorescence intensities of calibrator-signal substance compounds of different concentrations.

25. The mobile phone-based biological testing method according to claim 18, further comprising: a long-pass filter arranged in front of a camera of the mobile phone and configured to filter the excitation light.

26. The mobile phone-based biological testing method according to claim 25, wherein a process of calculating the concentration of each of the to-be-tested biomarkers according to the fluorescence intensity specifically comprises:
interpolating a calibration curve using the fluorescence intensity to obtain the concentration of each of the to-be-tested biomarkers, wherein the calibration curve is established according to fluorescence intensities of calibrator-signal substance compounds of different concentrations.

27. The mobile phone-based biological testing method according to claim 18, further comprising: a fluorescent immunochromatographic test strip configured to place the to-be-tested sample and comprising multiple control lines.

28. The mobile phone-based biological testing method according to claim 27, further comprising: a cassette, wherein the fluorescent immunochromatographic test strip is placed in the cassette; and the mobile phone is placed in or on the cassette.

29. The mobile phone-based biological testing method according to claim 18, wherein a process of calculating the concentration of each of the to-be-tested biomarkers according to the fluorescence intensity specifically comprises:
interpolating a calibration curve using the fluorescence intensity to obtain the concentration of each of the to-be-tested biomarkers, wherein the calibration curve is established according to fluorescence intensities of calibrator-signal substance compounds of different concentrations.

30. The mobile phone-based biological testing method according to claim 18, wherein the signal substances are quantum dots or time-resolved fluorescent microspheres.
